(19) United States
(12) Patent Application Publication
(10) Pub. No.: US 2007/0049975 A1
(43) Pub. Date: Mar. 1, 2007

(54) Active can with dedicated defibrillation and sensing electrodes

(76) Inventors: Adam W. Cates, Minneapolis, MN (US); Curtis Charles Lindstrom, Roseville, MN (US)

Correspondence Address: HOLLINGSWORTH & FUNK, LLC, Suite 125, 8009 34th Avenue South, Minneapolis, MN, 55425, US

(21) Appl. No.: 11/218,383

(22) Filed: Sep. 1, 2005

(51) Int. Cl. A61N 1/39

(52) U.S. Cl. 607/5

(57) ABSTRACT

Cardiac devices employing an active can and/or electrode assembly with dedicated defibrillation and sensing electrodes. A housing is configured for subcutaneous non-intrathoracic placement in a patient, the housing having a first face and a second face. One of the first and second face is directed away from the patient's heart, and the other of the first and second face is directed towards the patient's heart. A cardiac arrhythmia may be sensed using an electrode assembly positioned in or on the first face of the housing. A cardiac defibrillation therapy may be delivered to the patient's heart using an electrode assembly positioned in or on the second face of the housing in response to the detected arrhythmia.

### FIELD OF THE INVENTION

The present invention relates generally to implantable cardiac medical devices and, more particularly, to cardiac sensing and/or stimulation devices employing an active can and/or electrode assembly having dedicated defibrillation and sensing electrodes.

### BACKGROUND OF THE INVENTION

The healthy heart produces regular, synchronized contractions. Rhythmic contractions of the heart are normally initiated by the sinoatrial (SA) node, which is a group of specialized cells located in the upper right atrium. The SA node is the normal pacemaker of the heart, typically initiating 60-100 heartbeats per minute. When the SA node is pacing the heart normally, the heart is said to be in normal sinus rhythm.

If the heart's electrical activity becomes uncoordinated or irregular, the heart is denoted to be arrhythmic. Cardiac arrhythmia impairs cardiac efficiency and may be a potential life-threatening event. Cardiac arrhythmias have a number of etiological sources, including tissue damage due to myocardial infarction, infection, or degradation of the heart's ability to generate or synchronize the electrical impulses that coordinate contractions.

When the heart rate is too rapid, the condition is denoted as tachycardia. Tachycardia may have its origin in either the atria or the ventricles. Tachycardias occurring in the atria of the heart, for example, include atrial fibrillation and atrial flutter. Both conditions are characterized by rapid contractions of the atria. Besides being hemodynamically inefficient, the rapid contractions of the atria may also adversely affect the ventricular rate.

Ventricular tachycardia occurs, for example, when electrical activity arises in the ventricular myocardium at a rate more rapid than the normal sinus rhythm. Ventricular tachycardia may quickly degenerate into ventricular fibrillation. Ventricular fibrillation is a condition denoted by extremely rapid, uncoordinated electrical activity within the ventricular tissue. The rapid and erratic excitation of the ventricular tissue prevents synchronized contractions and impairs the heart's ability to effectively pump blood to the body, which is a fatal condition unless the heart is returned to sinus rhythm within a few minutes.

Implantable cardiac rhythm management systems have been used as an effective treatment for patients with serious arrhythmias. These systems typically include one or more leads and circuitry to sense signals from one or more interior and/or exterior surfaces of the heart. Such systems also include circuitry for generating electrical pulses that are applied to cardiac tissue at one or more interior and/or exterior surfaces of the heart. For example, leads extending into the patient's heart are connected to electrodes that contact the myocardium for sensing the heart's electrical signals and for delivering pulses to the heart in accordance with various therapies for treating arrhythmias.

Typical Implantable cardioverter/defibrillators (ICDs) include one or more leads to which at least one defibrillation electrode is connected. Such ICDs are capable of delivering high-energy shocks to the heart, interrupting the ventricular tachyarrhythmia or ventricular fibrillation, and allowing the heart to resume normal sinus rhythm.

### SUMMARY OF THE INVENTION

The present invention is directed to implantable cardiac medical devices and, more particularly, to cardiac sensing and/or stimulation devices employing an active can and/or electrode assembly having dedicated defibrillation and sensing electrodes. In one embodiment according to the present invention, an implantable cardiac device includes a housing having a first face, a second face, and an edge around the perimeter of the first face and extending from the first face to the perimeter of the second face. A pulse generator having a controller is provided in the housing. Two or more electrode assemblies are coupled to the pulse generator. The electrode assemblies include a first electrode assembly coupled to the housing and configured to sense a cardiac signal, and a second electrode assembly coupled to the housing and configured to deliver a defibrillation pulse. An insulating material electrically insulates the first electrode assembly from the second electrode assembly.

In another embodiment, the first electrode assembly and the second electrode assembly are arranged on opposite sides of the housing. For example, the first electrode assembly may include a housing electrode, and the second electrode assembly may be provided in or on the insulating material, wherein the insulating material is configured to matingly attach to the housing. In another example embodiment, the housing is configured as a curved elongated structure, a convex portion of the curved elongated structure defining the first face, and a concave portion of the curved elongated structure defining the second face, wherein the first electrode assembly is provided in or on the first face of the housing, and the second electrode assembly is provided in or on the second face of the housing.

Further embodiments in accordance with the present invention are directed to an implantable cardiac stimulation device having a housing configured for subcutaneous non-intrathoracic placement in a patient, the housing having a first face and a second face. Energy delivery circuitry is provided in the housing along with detection circuitry. At least one electrode arrangement is in or on the second face and coupled to the energy delivery circuitry, and at least one electrode arrangement is in or on the first face and coupled to the detection circuitry. A processor is provided in the housing and coupled to the energy delivery and detection circuitry, the processor configured to detect an arrhythmia using a cardiac signal developed from the first face electrode arrangement, the processor further configured to deliver a therapy that treats the arrhythmia using the second face electrode arrangement. In still further embodiments, the housing is configured as a curved elongated structure, a convex portion of the curved elongated structure defining the first face, and a concave portion of the curved elongated structure defining the second face.

Embodiments of methods in accordance with the present invention involve providing a housing configured for subcutaneous non-intrathoracic placement in a patient, the housing having a first face and a second face, wherein one of the first and second face is directed away from the patient's heart, and the other of the first and second face is directed towards the patient's heart. A cardiac arrhythmia may be sensed using an electrode assembly positioned in or on the first face of the housing. A cardiac defibrillation therapy may be delivered to the patient's heart using an electrode assembly positioned in or on the second face of the housing in response to the detected arrhythmia. Other embodiments of methods in accordance with the present invention involve mating an insulating cap to the first face of the housing, the insulating cap including the sensing electrode assembly.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1A is a top view of a cardiac device in accordance with the present invention;

FIG. 1B is a bottom view of a cardiac device in accordance with the present invention;

FIG. 1C is a top view of a cardiac device having side wrap-around electrodes in accordance with embodiments of the present invention;

FIG. 1D is a bottom view of a cardiac device having side wrap-around electrodes in accordance with the embodiment illustrated in FIG. 1C;

FIG. 1E is a side view of a cardiac device having side wrap-around electrodes in accordance with the embodiment illustrated in FIG. 1C;

FIG. 2 is a block diagram illustrating various processing and detection components of a cardiac device in accordance with an embodiment of the present invention;

FIG. 3A is a side view of a cardiac device housing mating to an insulating material having dedicated electrodes in accordance with embodiments of the present invention;

FIG. 3B is a top view of the insulating material having dedicated electrodes in accordance with the embodiment illustrated in FIG. 3A;

FIG. 4 is an illustration of an implantable cardiac device including a lead assembly shown implanted in a sectional view of a heart, in accordance with embodiments of the invention;

FIG. 5 is a diagram illustrating components of a cardiac stimulation device including an electrode assembly in accordance with an embodiment of the present invention; and

FIG. 6 is a block diagram illustrating various components of a cardiac device in accordance with an embodiment of the present invention.

While the invention is amenable to various modifications and alternative forms, specifics thereof have been shown by way of example in the drawings and will be described in detail below. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION OF VARIOUS EMBODIMENTS

In the following description of the illustrated embodiments, references are made to the accompanying drawings, which form a part hereof, and in which is shown by way of illustration, various embodiments in which the invention may be practiced. It is to be understood that other embodiments may be utilized, and structural and functional changes may be made without departing from the scope of the present invention.

An implanted device according to the present invention may include one or more of the features, structures, methods, or combinations thereof described hereinbelow. For example, a cardiac stimulator may be implemented to include one or more of the advantageous features and/or processes described below. It is intended that such a stimulator, or other implanted or partially implanted device need not include all of the features described herein, but may be implemented to include selected features that provide for unique structures and/or functionality. Such a device may be implemented to provide a variety of therapeutic or diagnostic functions.

A wide variety of implantable cardiac stimulation devices may be configured to implement dedicated active can defibrillation and sense electrodes of the present invention. A non-limiting, representative list of such devices includes cardiac monitors, pacemakers, cardiovertors, defibrillators, resynchronizers, and other cardiac monitoring and therapy delivery devices. These devices may be configured with a variety of electrode arrangements, including transvenous, endocardial, and epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes).

Embodiments of the present invention may be implemented in the context of a wide variety of cardiac devices, such as those listed above, and are referred to herein generally as patient-internal medical devices (PIMD) for convenience. A PIMD implemented in accordance with the present invention may incorporate dedicated defibrillation and sense electrodes along with one or more of the electrode types identified above and/or combinations thereof.

Cardiac devices employing dedicated defibrillation and sense electrodes of the present invention employ more than two electrodes of varying location, and possibly of varying configuration. Metal cans that house electronics for implantable cardiac stimulation devices may be employed as electrodes for sensing and for defibrillation. Also, indifferent electrodes that are electrically isolated from the can surface may be included on the header and/or can of the cardiac devices.

Embodiments of the present invention are directed to dedicated electrodes that serve as part of an implantable cardiac stimulation system. In one embodiment, the can may serve as a common electrode or a dedicated electrode, for the implantable cardiac stimulation system. In other embodiments, the electrodes may be provided on a substrate that has two distinct sides (e.g. a circular disk, a square or rectangular plate, or other shaped substrate). In yet another embodiment, the electrodes are provided in or on a unitary device.

In other embodiments of devices in accordance with the present invention, one side of the can includes a conductive surface that serves as the defibrillation electrode. Surface treatments or additional material affixed to the surface may be used to modify current flow across the electrode. This same side may also be used as a sensing electrode or as an auxiliary electrode to a sensing channel for noise reduction. On the other side of the can, one conductor or an arrangement of multiple conductors may be provided for subcutaneous electrocardiogram signal collection. Other sensors, such as photoplethysmography or acoustic sensors, may also be provided on that side. The rest of the can's surface may be non-conducting.

In further embodiments, a hermetically sealed can that is conductive may serve as the defibrillation electrode. On one side, a nonconductive external surface with sense electrodes embedded in it may be attached.

Embodiments of devices in accordance with the present invention may be placed subcutaneously with the defibrillation electrode side towards the fascia and skeletal muscle and the sensing side towards the skin. The device may be located in the pectoral region, over the ribs, in the subxiphoid region, or it may be directly over the heart. Other embodiments may be placed subcutaneously with the sensing side towards the fascia and skeletal muscle and the defibrillation electrode side towards the skin.

Devices in accordance with the present invention simplify the use of a subcutaneous system by incorporating multiple electrodes onto a single, compact object that may be easy to implant and minimize patient discomfort. In addition, the performance of each electrode may be improved. By facing the defibrillation electrode towards the fascia, current may be directed towards the heart. At the same time, sensing electrodes may improve the ratio of cardiac electrical signal to skeletal muscle electrical signal by eliminating direct contact with the skeletal muscle and increasing the distance of the electrodes from the skeletal muscle, while insignificantly reducing the distance of the electrodes from the heart. Also, the defibrillation electrode may serve as a reference electrode that can be used to eliminate or reduce much of the skeletal muscle signal.

The combination of dedicated electrodes in accordance with the present invention provides a solution for integrating separate sensing and defibrillation electrodes onto one device that may be helpful for a subcutaneous only cardiac stimulation system. For example, if both sensing and defibrillation electrodes are most effective over the apex of the heart, the present invention may allow coincidental placement of the electrodes in a manner that does not impact, and may even improve, performance of each electrode.

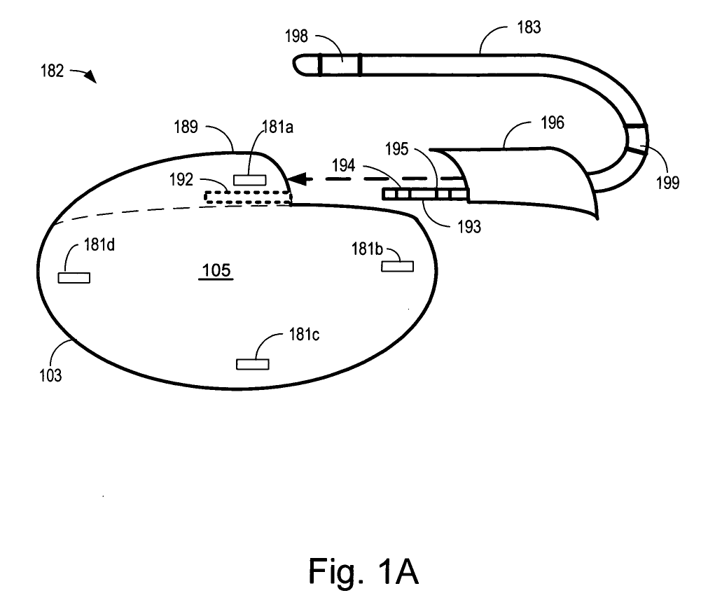

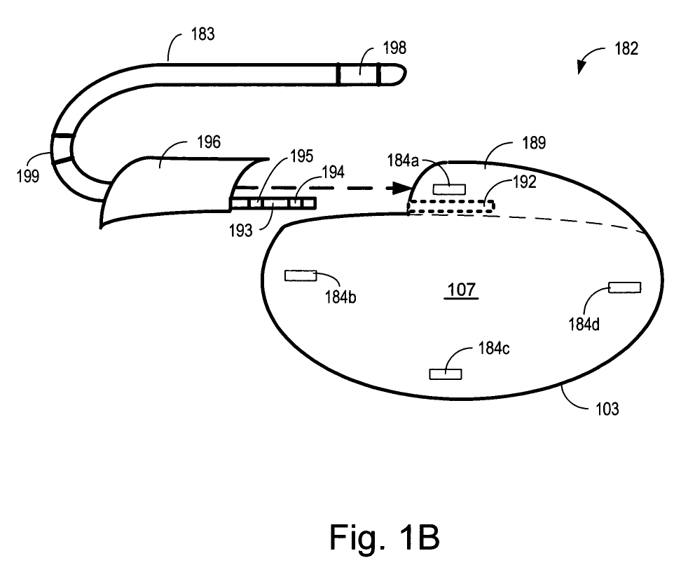

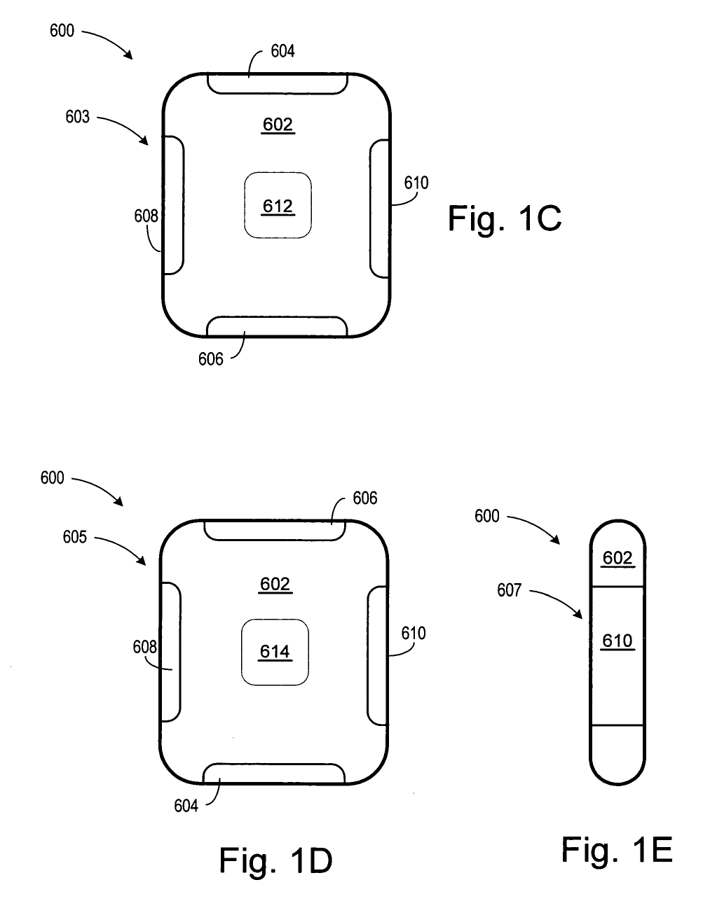

FIGS. 1A and 1B are top and bottom views respectively of a PIMD device 182 in accordance with the present invention, having at least one dedicated electrode on each face of a can 103. The PIMD device 182 may also include lead electrodes, such as a first electrode 198 and a second electrode 199 coupled to the can 103 through a header 189, via an electrode module 196. The first electrode 198 and second electrode 199 may be located on a lead 183 (single or multiple lead, or electrode array), or may be located directly in or on the electrode module 196.

The can 103 is illustrated as incorporating the header 189. The header 189 may be configured to facilitate removable attachment between an electrode module 196 and the can 103, as is shown in the embodiment depicted in FIGS. 1A and 1B. The header 189 includes a female coupler 192 configured to accept a male coupler 193 from the electrode module 196. The male coupler 193 is shown having two electrode contacts 194, 195 for coupling one or more electrodes 198 through the electrode module 196 to the can 103. An electrode 181a is illustrated on the header 189 relative to the top face of the can 103 in FIG. 1A, and an electrode 184a is illustrated on the header 189 relative to the bottom face of the can 103 in FIG. 1B. The can 103 is illustrated in FIGS. 1A and 1B having electrodes 181b, 181c, and 181d positioned on a top face 105 of the can 103 (FIG. 1A) and electrodes 184b, 184c, and 184d positioned on a bottom face 107 of the can 103 (FIG. 1B). The terms top and bottom are used for descriptive purposes only, and not as limitations to positioning.

In this and other configurations, the header 189 incorporates interface features (e.g., electrical connectors, ports, engagement features, and the like) that facilitate electrical connectivity with one or more lead and/or sensor systems, lead and/or sensor modules, and electrodes. The interface features of the header 189 may be protected from body fluids using known techniques.

The PIMD device 182 may further include one or more sensors in or on the can 103, header 189, electrode module 196, or lead(s) that couple to the header 189 or electrode module 196. Useful sensors may include electrophysiologic and non-electrophysiologic sensors, such as an acoustic sensor, an impedance sensor, a blood sensor, such as an oxygen saturation sensor (oximeter or plethysmographic sensor), a blood pressure sensor, minute ventilation sensor, or other sensors described or incorporated herein. Devices and methods for sensing blood oxygen are further described in commonly owned co-pending U.S. patent application Ser. No. 10/817,749, which is hereby incorporated herein by reference.

FIGS. 1C, 1D, and 1E are top, bottom, and side views respectively of a PIMD 600 having dedicated electrodes and side wrap-around electrodes in accordance with embodiments of the present invention. Although the PIMD 600 is illustrated as generally rectangular in shape, the PIMD 600 may be generally round, generally oval, generally triangular, generally square, generally pentagonal, generally hexagonal, or other shape without departing from the scope of the present invention.

The PIMD 600 includes a housing 602 that may house the componentry generally associated with a cardiac therapy device. The PIMD 600 includes a top face 603 (FIG. 1C), a bottom face 605 (FIG. 1D), and at least one side 607 (FIG. 1E). The PIMD is illustrated as having multiple wrap-around electrodes, which wrap from the front, around the side, and onto the back of the PIMD 600. An example of one type of wrap-around electrode is an electrode 610, which is illustrated as wrapping from the top face 603 in FIG. 1C, around the side 607 in FIG. 1E, and on the bottom face 605 in FIG. 1D.

Similarly, electrodes 604, 606, and 608 are illustrated in FIGS. 1C and 1D wrapping from the top face 603 to the bottom face 605; As stated previously, the terms top and bottom are intended as useful descriptors for illustrative purposes, and not intended to limit the actual use or orientation of the PIMD 600. In addition to the electrodes 604, 606, 608, and 610, a dedicated top face electrode 612 is illustrated on the top face 603 in FIG. 1C, and a dedicated bottom face electrode 614 is illustrated on the bottom face 605 in FIG. 1D. For example, Electrodes 608 and 612 on the top face 603 may be associated with cardiac signal sensing, and electrodes 608 and 614 on the bottom face 605 may be associated with the cardiac therapy delivery if the PIMD 600 is implanted in a patient with the bottom face 605 facing the patient's heart and the top face 603 facing the patient's skin, in one embodiment.

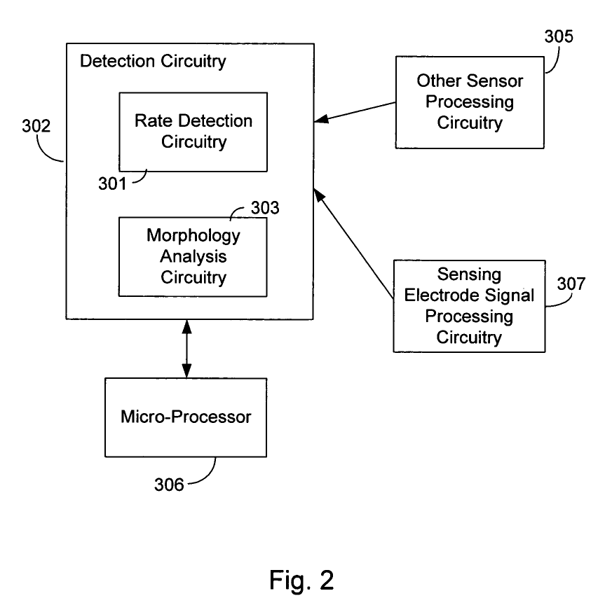

FIG. 2 illustrates a configuration of detection circuitry 302 of a PIMD, which includes one or both of rate detection circuitry 301 and morphological analysis circuitry 303. Detection and verification of arrhythmias may be accomplished using rate-based discrimination algorithms as known in the art implemented by the rate detection circuitry 301. Arrhythmic episodes may also be detected and verified by morphology-based analysis of sensed cardiac signals as is known in the art. Tiered or parallel arrhythmia discrimination algorithms may also be implemented using both rate-based and morphologic-based approaches. Further, a rate and pattern-based arrhythmia detection and discrimination approach may be employed to detect and/or verify arrhythmic episodes, such as the approach disclosed in U.S. Pat. Nos. 6,487,443; 6,259,947; 6,141,581; 5,855,593; and 5,545,186, which are hereby incorporated herein by reference.

The detection circuitry 302, which is coupled to a microprocessor 306, may be configured to incorporate, or communicate with, specialized circuitry for processing sensed cardiac signals in manners particularly useful in a cardiac sensing and/or stimulation device. As is shown by way of example in FIG. 2, the detection circuitry 302 may receive information from multiple physiologic and non-physiologic sensors, processed through other sensor processing circuitry 305.

The detection circuitry 302 may also receive information from one or more dedicated electrodes that sense cardiac activity. Processing circuitry 307 receives signals from one or more dedicated sensing electrodes, and transmits processed signal data to the detection circuitry 302. This data may be used to discriminate normal cardiac sinus rhythm from cardiac arrhythmias.

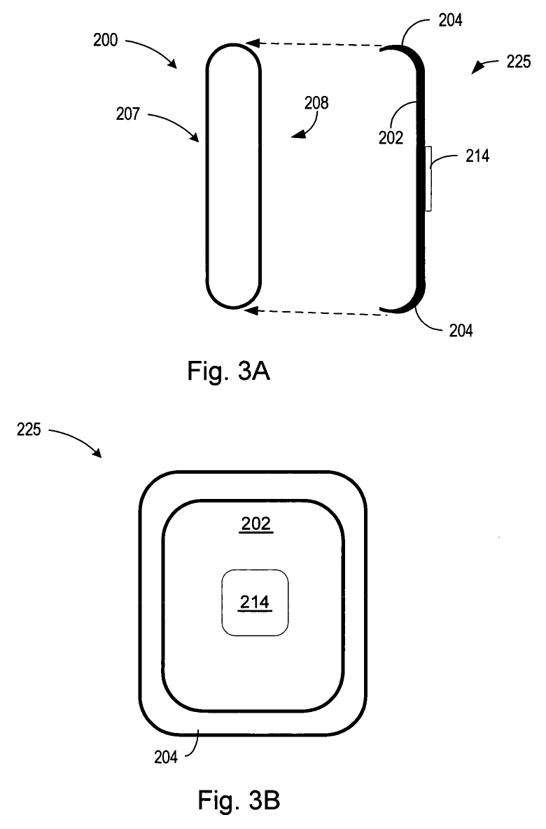

FIG. 3A is a side view of a cardiac device housing 200 matable to an insulating material 202 having dedicated electrodes in accordance with embodiments of the present invention. The cardiac device housing 200 is illustrated in side view, having a first side 207 and a second side 208. The cardiac device housing 200, in the embodiment illustrated in FIG. 3A is provided as an active can, where the entire body of the cardiac device housing 200 is conductive. The insulating material 202 is matable to the cardiac device housing 200 as illustrated by the dashed lines. The insulating material 202 may be provided as a cap that may be pressed onto the cardiac device housing 200 to mate, or the insulating material 202 may be formed onto the cardiac device housing 200 to mate with the cardiac device housing 200, such as by insert-molding or the like. For example, the insulating material 202 may be sprayed onto the cardiac device housing 200, or the cardiac device housing 200 may be dipped into the insulating material 202 in a liquid form.

One or more dedicated electrodes may be provided using the insulating material 202 in accordance with the present invention. For example, a center electrode 214 and an annular electrode 204 are illustrated in FIGS. 3A and 3B incorporated into the insulating material 202. The center electrode 214 and the annular electrode 204 may be molded into the insulating material 202, or otherwise attached to the insulating material 202.

FIG. 3B is a top view of the insulating material 202 having dedicated electrodes in accordance with the embodiment illustrated in FIG. 3A. In FIG. 3B, the annular electrode 204 is illustrated as surrounding the entire periphery of the insulating material 202, and wrapping around the sides of the insulating material 202. The center electrode 214 is illustrated at the center of the top face of the insulating material 202. The insulating material 202, after mating to the cardiac device housing 200, provides dedicated electrodes in accordance with the present invention. The insulating material 202 may be mated, in this example, to either the first face 207 or the second face 208 of the cardiac device housing 200 illustrated in FIG. 3A.

For purposes of illustration, and not of limitation, various embodiments of devices that may use dedicated defibrillation and sense electrodes in accordance with the present invention are described herein in the context of PIMD's that may be implanted under the skin in the chest region of a patient. A PIMD may, for example, be implanted subcutaneously such that all or selected elements of the device are positioned on the patient's front, back, side, or other body locations suitable for monitoring cardiac activity and delivering cardiac stimulation therapy. It is understood that elements of the PIMD may be located at several different body locations, such as in the chest, abdominal, or subclavian region with electrode elements respectively positioned at different regions near, around, in, or on the heart.

The primary housing (e.g., the active or non-active can) of the PIMD, for example, may be configured for positioning outside of the rib cage at an intercostal or subcostal location, within the abdomen, or in the upper chest region (e.g., subclavian location, such as above the third rib). In one implementation, one or more leads incorporating electrodes may be located in direct contact with the heart, great vessels or coronary vasculature, such as via one or more leads implanted by use of conventional transvenous delivery approaches. In another implementation, one or more electrodes may be located on the primary housing and/or at other locations about, but not in direct contact with the heart, great vessels or coronary vasculature.

In a further implementation, for example, one or more electrode subsystems or electrode arrays may be used to sense cardiac activity and deliver cardiac stimulation energy in a PIMD configuration employing an active can or a configuration employing a non-active can. Electrodes may be situated at anterior and/or posterior locations relative to the heart. Examples of useful electrode locations and features that may be incorporated in various embodiments of the present invention are described in commonly owned, co-pending U.S. patent application Ser. No. 10/465,520 filed Jun. 19, 2003, entitled “Methods and Systems Involving Subcutaneous Electrode Positioning Relative to a Heart”; Ser. No. 10/795,126 filed Mar. 5, 2004, entitled “Wireless ECG In Implantable Devices”; and Ser. No. 10/738,608 filed Dec. 17, 2003, entitled “Noise Canceling Cardiac Electrodes,” which are hereby incorporated herein by reference.

Configurations of PIMDs in accordance with the present invention are illustrated herein as capable of implementing various functions of a cardioverter/defibrillator (ICD), and may operate in numerous cardioversion/defibrillation modes as are known in the art. Examples of ICD circuitry, structures and functionality, aspects of which may be incorporated in a PIMD of a type that may benefit from dedicated defibrillation and sensing electrodes are disclosed in commonly owned U.S. Pat. Nos. 5,133,353; 5,179,945; 5,314,459; 5,318,597; 5,620,466; and 5,662,688, which are hereby incorporated herein by reference.

A PIMD may be used to implement various diagnostic functions, which may involve performing rate-based, pattern and rate-based, and/or morphological tachyarrhythmia discrimination analyses. Subcutaneous, cutaneous, and/or external sensors may be employed to acquire physiologic and non-physiologic information for purposes of enhancing tachyarrhythmia detection and termination. It is understood that configurations, features, and combination of features described in the present disclosure may be implemented in a wide range of implantable medical devices, and that such embodiments and features are not limited to the particular devices described herein.

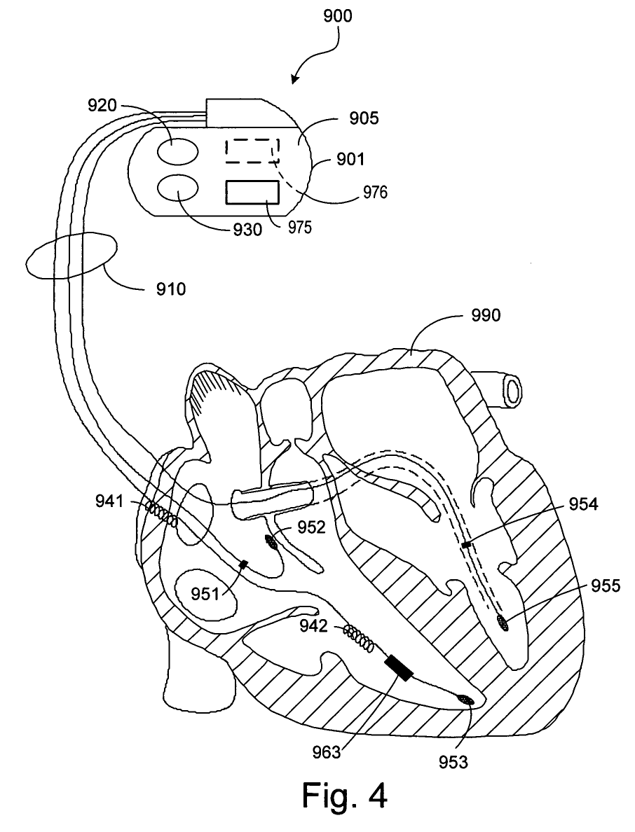

Referring now to FIG. 4, the implantable device illustrated in FIG. 4 is an embodiment of a PIMD having dedicated defibrillation and sense electrodes in accordance with the present invention. In this example, the implantable device includes a cardiac rhythm management device (CRM) 900 including an implantable pulse generator 905 electrically and physically coupled to an intracardiac lead system 910. The CRM 900 includes a dedicated first electrode 975 on the front face, and a dedicated second electrode 976 on the back face in accordance with an embodiment of the present invention.

Portions of the intracardiac lead system 910 are inserted into the patient's heart 990. The intracardiac lead system 910 includes one or more electrodes configured to sense electrical cardiac activity of the heart, deliver electrical stimulation to the heart, sense the patient's transthoracic impedance, and/or sense other physiological parameters, e,g, cardiac chamber pressure or temperature. Portions of the housing 901 of the pulse generator 905 may optionally serve as a can electrode.

Communications circuitry is disposed within the housing 901 for facilitating communication between the pulse generator 905 and an external communication device, such as a portable or bed-side communication station, patient-carried/worn communication station, or external programmer, for example. The communications circuitry may also facilitate unidirectional or bidirectional communication with one or more implanted, external, cutaneous, or subcutaneous physiologic or non-physiologic sensors, patient-input devices and/or information systems.

The pulse generator 905 may optionally incorporate a motion detector 920 that may be used to sense patient activity as well as various respiration and cardiac related conditions. For example, the motion detector 920 may be optionally configured to sense snoring, activity level, and/or chest wall movements associated with respiratory effort, for example. The motion detector 920 may be implemented as an accelerometer positioned in or on the housing 901 of the pulse generator 905. If the motion sensor is implemented as an accelerometer, the motion sensor may also provide respiratory, e.g. rales, coughing, and cardiac, e.g. S1-S4 heart sounds, murmurs, and other acoustic information.

The lead system 910 and pulse generator 905 of the CRM 900 may incorporate one or more transthoracic impedance sensors that may be used to acquire the patient's respiration waveform, or other respiration-related information. The transthoracic impedance sensor may include, for example, one or more intracardiac electrodes 941, 942, 951-955, 963 positioned in one or more chambers of the heart 990. The intracardiac electrodes 941, 942, 951-955, 963 may be coupled to impedance drive/sense circuitry 930 positioned within the housing of the pulse generator 905.

The lead system 910 may include one or more cardiac pace/sense electrodes 951-955 positioned in, on, or about one or more heart chambers for sensing electrical signals from the patient's heart 990 and/or delivering pacing pulses to the heart 990. The intracardiac sense/pace electrodes 951-955, such as those illustrated in FIG. 4, may be used to sense and/or pace one or more chambers of the heart, including the left ventricle, the right ventricle, the left atrium and/or the right atrium. The lead system 910 may include one or more defibrillation electrodes 941, 942 for delivering defibrillation/cardioversion shocks to the heart.

The pulse generator 905 may include circuitry for detecting cardiac arrhythmias and for controlling pacing or defibrillation therapy in the form of electrical stimulation pulses or shocks delivered to the heart through the lead system 910. The pulse generator 905 may also incorporate circuitry, structures and functionality of the implantable medical devices disclosed in commonly owned U.S. Pat. Nos. 5,203,348; 5,230,337; 5,360,442; 5,366,496; 5,397,342; 5,391,200; 5,545,202; 5,603,732; and 5,916,243; 6,360,127; 6,597,951; and US Patent Publication No. 2002/0143264, which are hereby incorporated herein by reference.

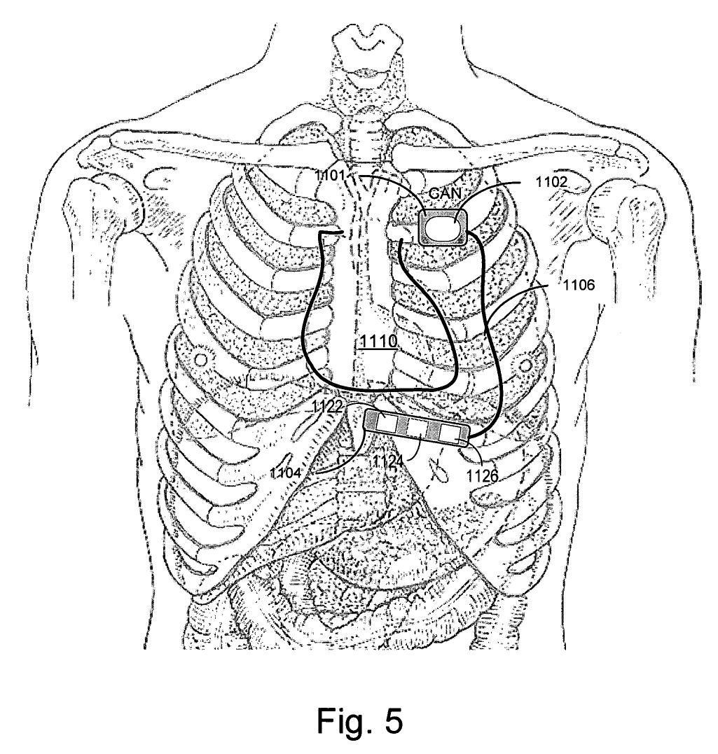

In one configuration, as is illustrated in FIG. 5, electrode subsystems of a PIMD system are arranged about a patient's heart 1110. The PIMD system includes a first electrode subsystem, including a can electrode 1102, and a second electrode assembly, designated electrode subsystem 1104, including dedicated electrodes in accordance with embodiments of the present invention. The second electrode subsystem 1104 may include any number of electrodes used for sensing and/or electrical stimulation and is connected to pulse generator 905 via lead 1106.

In various configurations, the second electrode subsystem 1104 may include a combination of dedicated electrodes. The combination of electrodes of the second electrode subsystem 1104 may include one or more of coil electrodes, tip electrodes, ring electrodes, multi-element coils, spiral coils, screen patch electrodes, circular disks or disk electrodes in other shapes, multiple electrodes arranged on non-conductive backings, and other electrode configurations as described herein or as incorporated by reference.

Dedicated electrodes, such as a first electrode 1122, a second electrode 1124, and a third electrode 1126 are provided on the second electrode subsystem 1104, which is a non-conductive backing for the dedicated electrodes 1122, 1124, and 1126. A suitable non-conductive backing material is silicone rubber, for example. The second electrode subsystem 1104, in the example illustrated in FIG. 5, includes the first electrode 1122 and the third electrode 1126 on a first surface, and the second electrode 1124 on a second surface. The second electrode 1124 is illustrated in FIG. 5 as facing toward the heart 1110, and the first electrode 1122 and third electrode 1126 are illustrated as facing away the heart 1110, towards the patient's skin.

The can electrode 1102 is positioned on the housing 1101 that encloses the PIMD electronics. In one embodiment, the can electrode 1102 includes the entirety of the external surface of housing 1101. In other embodiments, various portions of the housing 1101 may be electrically isolated from the can electrode 1102 or from tissue. For example, the active area of the can electrode 1102 may include all or a portion of either the anterior or posterior surface of the housing 1101 to direct current flow in a manner advantageous for cardiac sensing and/or stimulation.

Portions of the housing may be electrically isolated from tissue to optimally direct current flow. For example, portions of the housing 1101 may be covered with a non-conductive, or otherwise electrically resistive, material to direct current flow. Suitable non-conductive material coatings include those formed from silicone rubber, polyurethane, or parylene, for example.

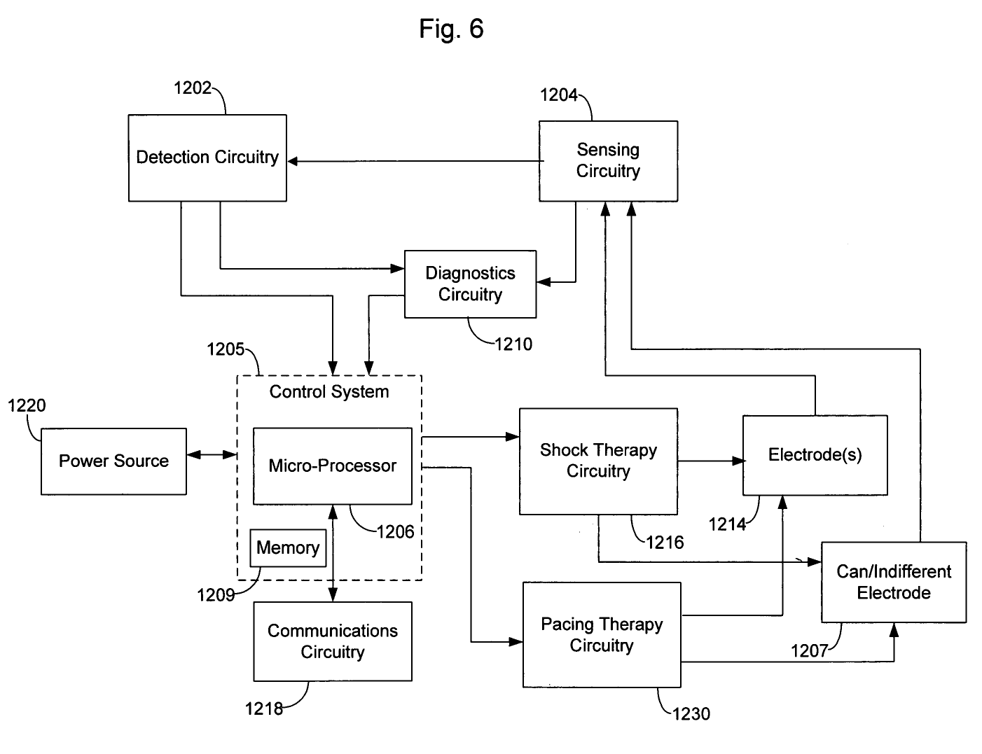

FIG. 6 is a block diagram depicting various componentry of different arrangements of a PIMD in accordance with embodiments of the present invention. The components, functionality, and configurations depicted in FIG. 6 are intended to provide an understanding of various features and combinations of features that may be incorporated in a PIMD. It is understood that a wide variety of device configurations are contemplated, ranging from relatively sophisticated to relatively simple designs. As such, particular PIMD configurations may include some componentry illustrated in FIG. 6, while excluding other componentry illustrated in FIG. 6.

Illustrated in FIG. 6 is a processor-based control system 1205 which includes a micro-processor 1206 coupled to appropriate memory (volatile and/or non-volatile) 1209, it being understood that any logic-based control architecture may be used. The control system 1205 is coupled to circuitry and components to sense, detect, and analyze electrical signals produced by the heart and deliver electrical stimulation energy to the heart under predetermined conditions to treat cardiac arrhythmias or/and other cardiac conditions. The control system 1205 and associated components also provide pacing therapy to the heart. The electrical energy delivered by the PIMD may be in the form of low energy pacing pulses or high-energy pulses for cardioversion or defibrillation.

Cardiac signals are sensed using the electrode(s) 1214 and the can or indifferent electrode 1207 provided on the PIMD housing. Cardiac signals may also be sensed using only the electrode(s) 1214, such as in a non-active can configuration. As such, unipolar, bipolar, or combined unipolar/bipolar electrode configurations as well as multi-element electrodes and combinations of noise canceling and standard electrodes may be employed. The sensed cardiac signals are received by sensing circuitry 1204, which includes sense amplification circuitry and may also include filtering circuitry and an analog-to-digital (A/D) converter.

Detection circuitry 1202 may include a signal processor that coordinates analysis of the sensed cardiac signals and/or other sensor inputs to detect cardiac arrhythmias, such as, in particular, tachyarrhythmia. Rate based and/or morphological discrimination algorithms may be implemented by the signal processor of the detection circuitry 1202 to detect and verify the presence and severity of an arrhythmic episode. Examples of arrhythmia detection and discrimination circuitry, structures, and techniques, aspects of which may be implemented by a PIMD of a type that may benefit from dedicated defibrillation and sensing electrode methods and implementations are disclosed in commonly owned U.S. Pat. Nos. 5,301,677, 6,438,410, and 6,708,058, which are hereby incorporated herein by reference.

The detection circuitry 1202 communicates cardiac signal information to the control system 1205. Memory circuitry 1209 of the control system 1205 contains parameters for operating in various monitoring, defibrillation, and, if applicable, pacing modes, and stores data indicative of cardiac signals received by the detection circuitry 1202. The memory circuitry 1209 may also be configured to store historical ECG and therapy data, which may be used for various purposes and transmitted to an external receiving device as needed or desired.

In certain configurations, the PIMD may include diagnostics circuitry 1210. The diagnostics circuitry 1210 typically receives input signals from the detection circuitry 1202 and the sensing circuitry 1204. The diagnostics circuitry 1210 provides diagnostics data to the control system 1205, it being understood that the control system 1205 may incorporate all or part of the diagnostics circuitry 1210 or its functionality. The control system 1205 may store and use information provided by the diagnostics circuitry 1210 for a variety of diagnostics purposes. This diagnostic information may be stored, for example, subsequent to a triggering event or at predetermined intervals, and may include system diagnostics, such as power source status, therapy delivery history, and/or patient diagnostics. The diagnostic information may take the form of electrical signals or other sensor data acquired immediately prior to therapy delivery.

According to a configuration that provides cardioversion and defibrillation therapies, the control system 1205 processes cardiac signal data received from the detection circuitry 1202 and initiates appropriate tachyarrhythmia therapies to terminate cardiac arrhythmic episodes and return the heart to normal sinus rhythm. The control system 1205 is coupled to shock therapy circuitry 1216. The shock therapy circuitry 1216 is coupled to the electrode(s) 1214 and the can or indifferent electrode 1207 of the PIMD housing.

Upon command, the shock therapy circuitry 1216 delivers cardioversion and defibrillation stimulation energy to the heart in accordance with a selected cardioversion or defibrillation therapy. In a less sophisticated configuration, the shock therapy circuitry 1216 is controlled to deliver defibrillation therapies, in contrast to a configuration that provides for delivery of both cardioversion and defibrillation therapies. Examples of PIMD high energy delivery circuitry, structures and functionality, aspects of which may be incorporated in a PIMD of a type that may benefit from aspects of the present invention are disclosed in commonly owned U.S. Pat. Nos. 5,372,606; 5,411,525; 5,468,254; and 5,634,938, which are hereby incorporated herein by reference.

Arrhythmic episodes may also be detected and verified by morphology-based analysis of sensed cardiac signals as is known in the art. Tiered or parallel arrhythmia discrimination algorithms may also be implemented using both rate-based and morphologic-based approaches. Further, a rate and pattern-based arrhythmia detection and discrimination approach may be employed to detect and/or verify arrhythmic episodes, such as the approach disclosed in U.S. Pat. Nos. 6,487,443; 6,259,947; 6,141,581; 5,855,593; and 5,545,186, which are hereby incorporated herein by reference.

In accordance with another configuration, a PIMD may incorporate a cardiac pacing capability in addition to cardioversion and/or defibrillation capabilities. As is shown in FIG. 6, the PIMD includes pacing therapy circuitry 1230 that is coupled to the control system 1205 and the electrode(s) 1214 and can/indifferent electrodes 1207. Upon command, the pacing therapy circuitry 1230 delivers pacing pulses to the heart in accordance with a selected pacing therapy.

The PIMD shown in FIG. 6 may be configured to receive signals from one or more physiologic and/or non-physiologic sensors. Depending on the type of sensor employed, signals generated by the sensors may be communicated to transducer circuitry coupled directly to the detection circuitry 1202 or indirectly via the sensing circuitry 1204. It is noted that certain sensors may transmit sense data to the control system 1205 without processing by the detection circuitry 1202.

Communications circuitry 1218 is coupled to the microprocessor 1206 of the control system 1205. The communications circuitry 1218 allows the PIMD to communicate with one or more receiving devices or systems situated external to the PIMD. By way of example, the PIMD may communicate with a patient-worn, portable or bedside communication system via the communications circuitry 1218. In one configuration, one or more physiologic or non-physiologic sensors (subcutaneous, cutaneous, or external of patient) may be equipped with a short-range wireless communication interface, such as an interface conforming to a known communications standard, such as Bluetooth or IEEE 802 standards. Data acquired by such sensors may be communicated to the PIMD via the communications circuitry 1218. It is noted that physiologic or non-physiologic sensors equipped with wireless transmitters or transceivers may communicate with a receiving system external of the patient.

The communications circuitry 1218 allows the PIMD to communicate with an external programmer. In one configuration, the communications circuitry 1218 and the programmer unit (not shown) use a wire loop antenna and a radio frequency telemetric link, as is known in the art, to receive and transmit signals and data between the programmer unit and communications circuitry 1218. In this manner, programming commands and data are transferred between the PIMD and the programmer unit during and after implant. Using a programmer, a physician is able to set or modify various parameters used by the PIMD. For example, a physician may set or modify parameters affecting monitoring, detection, pacing, and defibrillation functions of the PIMD, including pacing and cardioversion/defibrillation therapy modes.

Typically, the PIMD is encased and hermetically sealed in a housing suitable for implanting in a human body as is known in the art. Power to the PIMD is supplied by an electrochemical power source 1220 housed within the PIMD. In one configuration, the power source 1220 includes a rechargeable battery. According to this configuration, charging circuitry is coupled to the power source 1220 to facilitate repeated non-invasive charging of the power source 1220. The communications circuitry 1218, or separate receiver circuitry, is configured to receive RF energy transmitted by an external RF energy transmitter. The PIMD may, in addition to a rechargeable power source, include a non-rechargeable battery. It is understood that a rechargeable power source need not be used, in which case a long-life non-rechargeable battery is employed.

The detection circuitry 1202, which is coupled to a microprocessor 1206, may be configured to incorporate, or communicate with, specialized circuitry for processing sensed cardiac signals in manners particularly useful in a cardiac sensing and/or stimulation device. As is shown by way of example in FIG. 6, the detection circuitry 1202 may receive information from multiple physiologic and non-physiologic sensors.

The components, functionality, and structural configurations depicted herein are intended to provide an understanding of various features and combination of features that may be incorporated in a PIMD. It is understood that a wide variety of PIMDs and other implantable cardiac stimulation device configurations are contemplated, ranging from relatively sophisticated to relatively simple designs. As such, particular PIMD or cardiac stimulation device configurations may include particular features as described herein, while other such device configurations may exclude particular features described herein.

A PIMD of the present invention may be used within the structure of an advanced patient management (APM) system. The advanced patient management system allows physicians to remotely and automatically monitor cardiac and respiratory functions, as well as other patient conditions. In one example, a PIMD implemented as a cardiac pacemaker, defibrillator, or resynchronization device may be equipped with various telecommunications and information technologies that enable real-time data collection, diagnosis, and treatment of the patient. Various PIMD embodiments described herein may be used in connection with advanced patient management. Methods, structures, and/or techniques described herein, which may be adapted to provide for remote patient/device monitoring, diagnosis, therapy, or other APM related methodologies, may incorporate features of one or more of the following references: U.S. Pat. Nos. 6,221,011; 6,270,457; 6,277,072; 6,280,380; 6,312,378; 6,336,903; 6,358,203; 6,368,284; 6,398,728; and 6,440,066, which are hereby incorporated herein by reference.

Various modifications and additions can be made to the preferred embodiments discussed hereinabove without departing from the scope of the present invention. Accordingly, the scope of the present invention should not be limited by the particular embodiments described above, but should be defined only by the claims set forth below and equivalents thereof.

What is claimed is:

1. An implantable cardiac device, comprising:
a housing having a first face, a second face, and an edge around the perimeter of the first face and extending from the first face to the perimeter of the second face;
a pulse generator comprising a controller provided in the housing; and
a plurality of electrode assemblies coupled to the pulse generator, the plurality of electrode assemblies comprising:
a first electrode assembly coupled to the housing and configured to sense a cardiac signal;
a second electrode assembly coupled to the housing and configured to deliver a defibrillation pulse; and
an insulating material electrically insulating the first electrode assembly from the second electrode assembly;
wherein the first electrode assembly and the second electrode assembly are arranged on opposite sides of the housing.

2. The device of claim 1, wherein the first electrode assembly is a housing electrode, and the second electrode assembly is provided in or on the insulating material, wherein the insulating material is configured to matingly attach to the housing.

3. The device of claim 1, wherein the housing is configured as a curved elongated structure, a convex portion of the curved elongated structure defining the first face, and a concave portion of the curved elongated structure defining the second face, wherein the first electrode assembly is provided in or on the first face of the housing, and the second electrode assembly is provided in or on the second face of the housing.

4. The device of claim 1, wherein one or both of the first electrode assembly and the second electrode assembly comprises an electrode array.

5. The device of claim 1, wherein the second electrode assembly is a housing electrode, and the first electrode assembly comprises an electrode array provided in or on the insulating material, wherein the insulating material is configured to matingly attach to the housing.

6. The device of claim 5, wherein the insulating material is configured to cover the first face and edge of the housing.

7. An implantable cardiac stimulation device, comprising:
a housing configured for subcutaneous non-intrathoracic placement in a patient, the housing comprising a first face and a second face;
energy delivery circuitry provided in the housing;
detection circuitry provided in the housing;
at least one electrode arrangement in or on the second face and coupled to the energy delivery circuitry;
at least one electrode arrangement in or on the first face and coupled to the detection circuitry; and
a processor provided in the housing and coupled to the energy delivery and detection circuitry, the processor configured to detect an arrhythmia using a cardiac signal developed from the at least one first face electrode arrangement, the processor further configured to deliver a therapy that treats the arrhythmia using the at least one second face electrode arrangement.

8. The device of claim 7, wherein the housing is configured as a curved elongated structure, a convex portion of the curved elongated structure defining the first face, and a concave portion of the curved elongated structure defining the second face.

9. The device of claim 7, wherein one or both of the first electrode arrangement and the second electrode arrangement comprises an electrode array.

10. The device of claim 7, wherein the housing comprises an acoustic sensor positioned in or on the first face of the housing.

11. The device of claim 7, wherein the housing comprises a photoplethysmography sensor positioned in or on the first face of the housing.

12. The device of claim 7, wherein the housing comprises a non-electrophysiologic sensor positioned in or on the first face of the housing.

13. The device of claim 7, wherein one or both of the first face electrode arrangement and the second face electrode arrangement comprises a surface treatment configured to modify current flow across the electrode.

14. A method, comprising:
providing a housing configured for subcutaneous non-intrathoracic placement in a patient, the housing comprising a first face and a second face, wherein one of the first and second face is directed away from the patient's heart, and the other of the first and second face is directed towards the patient's heart;
sensing a cardiac arrhythmia using an electrode assembly positioned in or on the first face of the housing; and
delivering a cardiac defibrillation therapy to the patient's heart using an electrode assembly positioned in or on the second face of the housing in response to the detected arrhythmia.

15. The method of claim 14, comprising mating an insulating cap to the first face of the housing, the insulating cap comprising the sensing electrode assembly.

16. The method of claim 14, comprising mating an insulating cap to the second face of the housing, the insulating cap comprising the delivering electrode assembly.

17. The method of claim 14, comprising:
providing a non-electrophysiologic sensor in or on the first face of the housing:
sensing a non-electrophysiologic signal using the non-electrophysiologic sensor; and
discriminating a cardiac signal from a non-cardiac signal using the non-electrophysiologic signal.

18. A device, comprising:
means for sensing a cardiac signal from a patient's heart;
means for detecting an arrhythmia using the cardiac signal;
means for delivering a cardiac stimulation therapy to the patient's heart in response to the detecting means; and
means for insulating the sensing means from the delivering means;
wherein the means for sensing and the means for delivering are arranged on opposite sides of the insulating means.

19. The device of claim 18, comprising:
means for sensing a non-electrophysiologic signal coupled to the arrhythmia detection means.

20. The device of claim 18, comprising:
means for mating the insulating means to a housing of a subcutaneous non-intrathoracic patient implantable medical device, the patient implantable medical device housing the detecting means and the delivering means.

21. A cardiac lead system, comprising:
a lead body configured for subcutaneous non-intrathoracic placement in a patient;
a non-conductive backing coupled to the lead body, the non-conductive backing comprising a first face and a second face, wherein one of the first and second face is configured to be directed away from the patient's heart, and the other of the first and second face is configured to be directed towards the patient's heart;
a sensing electrode assembly configured to sense a cardiac signal, the sensing electrode assembly positioned in or on the first face of the non-conductive backing; and
a therapy electrode assembly configured to deliver a cardiac defibrillation therapy to the patient's heart, the therapy electrode assembly positioned in or on the second face of the non-conductive backing.
